(19) United States
(12) Patent Application Publication
(10) Pub. No.: US 2023/0184738 A1
(43) Pub. Date: Jun. 15, 2023

(54) DETECTING LAB SPECIMEN VIABILITY

(71) Applicant: Optum, Inc., Minnetonka, MN (US)

(72) Inventors: Komal Khatri, Cedar Park, TX (US); Jon Kevin Muse, Thompsons Station, TN (US); Lisa Jo L Abbo, Chicago, IL (US); Gregory J. Boss, Saginaw, MI (US)

(21) Appl. No.: 17/644,538

(22) Filed: Dec. 15, 2021

(51) Int. Cl. G01N 33/487; G06T 7/00; B04B 13/00; G06N 20/00; B04B 9/10; B04B 15/02

(52) U.S. Cl. CPC G01N 33/48707; G06T 7/0012; B04B 13/00; G06N 20/00; B04B 9/10; B04B 15/02; B04B 2013/006; G06T 2207/20081; G06T 2207/30004

(57) ABSTRACT

A centrifuge includes a chamber configured to contain a set of one or more samples, an image sensor configured to generate image data, and processing circuitry. The processing circuitry is configured to: initiate centrifugation of the set of samples about a central axis; obtain a set of image data generated by the image sensor during centrifugation of the set of samples; and for each respective sample of the set of samples, apply a machine learning model configured to generate, based on image data representative of the respective sample in the set of image data, a viability score for the respective sample; determine whether the viability score for the respective sample satisfies a viability condition; and output whether the viability score for the respective sample satisfies the viability condition.

### BACKGROUND

Lab samples may be centrifuged prior to testing. If the lab sample is not centrifuged or not centrifuged properly, the lab sample may not be viable for its intended use. For example, it may not be possible to test the lab sample, or the test may return improper results. Determining whether a lab specimen has been centrifuged (and centrifuged properly) based on human visual inspection may be difficult. For instance, different lab samples may have different appearances based on not only whether the lab samples were centrifuged, but also, for example, the presence of various pathologies.

### SUMMARY

The present disclosure describes techniques for automating the process of determining whether a lab sample (“sample”), such as a blood sample, has been centrifuged properly and is viable for its intended use. For example, processing circuitry may obtain image data generated by an image sensor during rotation of a set of samples in a centrifuge. For each respective sample of the set of samples, the processing circuitry may apply a machine learning model configured to generate a viability score indicative of a likelihood of the respective sample being viable for an intended use. In some examples, the viability score may be based on various properties of the lab samples, such as colors, shapes, textures, formation of separate regions, volume of each region, etc. The processing circuitry may determine, for each sample of the set of samples, whether the viability score for the sample satisfies a viability condition. The viability condition may be configured such that samples with viability scores that satisfy the viability condition are (likely to be) viable for their intended use, and samples with viability scores that do not satisfy the viability condition are not (likely to be) viable for their intended use. The processing circuitry may then output, for each sample, whether the respective viability score for the sample satisfies the viability condition. In this way, the processing circuitry may indicate which samples are viable and which samples are not.

In some examples, a method comprises: initiating, by processing circuitry, centrifugation of a set of one or more samples within a chamber of a centrifuge and about a central axis; obtaining, by the processing circuitry, a set of image data generated by an image sensor during centrifugation of the set of samples, wherein the set of image data is representative of the set of samples; and for each respective sample of the set of samples: applying, by processing circuitry, a machine learning model configured to generate, based on image data representative of the respective sample in the set of image data, a viability score for the respective sample, wherein the viability score is indicative of a likelihood of the respective sample being viable for an intended use; determining, by the processing circuitry, whether the viability score for the sample satisfies a viability condition; and outputting, by the processing circuitry, a notification indicating whether the viability score for the sample satisfies the viability condition.

In some examples, a centrifuge comprises: a chamber configured to contain a set of one or more samples; an image sensor configured to generate image data; and processing circuitry configured to: initiate centrifugation of the set of samples about a central axis; obtain a set of image data generated by the image sensor during centrifugation of the set of samples, wherein the set of image data is representative of the set of samples; and for each respective sample of the set of samples: apply a machine learning model configured to generate, based on image data representative of the respective sample in the set of image data, a viability score for the respective sample, wherein the viability score is indicative of a likelihood of the respective sample being viable for an intended use; determine whether the viability score for the respective sample satisfies a viability condition; and output a notification indicating whether the viability score for the respective sample satisfies the viability condition.

The details of one or more aspects of the disclosure are set forth in the accompanying drawings and the description below. Other features, objects, and advantages of the techniques described in this disclosure will be apparent from the description, drawings, and claims.

### BRIEF DESCRIPTION OF THE DRAWINGS

FIG. 1 is a conceptual diagram illustrating an example system in accordance with one or more aspects of this disclosure.

FIG. 2 is a block diagram illustrating example components of a centrifuge in accordance with one or more aspects of this disclosure.

FIGS. 3A-B are conceptual diagrams illustrating example lab samples in accordance with one or more aspects of this disclosure.

FIGS. 4A-B are conceptual diagrams illustrating an example retention assembly of a centrifuge in accordance with one or more aspects of this disclosure.

FIG. 5 is a conceptual diagram illustrating an example retention assembly of a centrifuge in accordance with one or more aspects of this disclosure.

FIG. 6 is a conceptual diagram illustrating an example ejection mechanism in accordance with one or more aspects of this disclosure.

FIG. 7 is a flowchart illustrating an example method in accordance with one or more techniques of this disclosure.

### DETAILED DESCRIPTION

As part of preparing a lab sample (“sample”) for transportation, analysis, etc., personnel, such as a lab technician, may centrifuge the sample. Centrifugation is a technique that helps to separate mixtures by applying centrifugal force. Although the concept of centrifugation may seem straightforward, several issues may occur that in turn affect the viability of the sample for its intended use. For example, the content of a sample may render the sample not viable for its intended use, irrespective of the amount of centrifugation performed. In another example, a centrifuge may abort centrifugation prior to the sample properly separating because the centrifuge is unbalanced. In yet another example, the centrifuge may spin the sample at a predetermined speed and for a predetermined duration, but the predetermined duration may be insufficient such that the sample is still not properly separated. In yet another example, a lab technician may just accidentally skip “spinning down” the samples in the centrifuge.

These problems described above as well others may interrupt the process of preparing samples for transport, analysis, etc. Some of the existing solutions to these problems may be entirely human-reliant (e.g., require visual identification of viability). However, visually identifying whether a sample is viable post-centrifugation may require a high degree of skill and thus may not be readily available. Accordingly, more automated techniques may not only expedite the analytical process, but also allow for more accurate determinations of sample viability (which, in some instances, a human expert may review to ensure accuracy).

This disclosure describes techniques that may address one or more of these issues. As described herein, processing circuitry may obtain image data generated by an image sensor during rotation of a set of one or more samples in a centrifuge. For each respective sample of the set of samples, the processing circuitry may apply a machine learning model configured to generate a viability score indicative of a likelihood of the respective sample being viable for an intended use. In some examples, the viability score may be based on various properties of the respective sample, such as colors, shapes, textures, formation of separate regions, volume of each region, etc. The processing circuitry may determine, for each sample of the set of samples, whether the viability score for the sample satisfies a viability condition. The viability condition may be selected such that samples with viability scores that satisfy the viability condition are (likely to be) viable for their intended use, and samples with viability scores that do not satisfy the viability condition are not (likely to be) viable for their intended use. The processing circuitry may then output, for each sample, a notification indicating whether the viability score for the sample satisfies the viability condition. In this way, the processing circuitry may indicate which samples are viable and which samples are not.

In some examples, the processing circuitry may control a retention assembly of the centrifuge to adjust a center of gravity of the set of samples relative to a central axis about which the set of samples rotate. The retention assembly may include an inner ring and an outer ring, each configured to receive at least one sample of the set of samples. In such implementations, the processing circuitry may control the retention assembly by rotating at least one of the inner ring or the outer ring about the central axis, thereby repositioning samples in the inner ring or outer ring and adjusting the center of gravity of the set of samples relative to the central axis.

In some examples, the viability score for a sample may be based, at least in part, on a degree of region separation of the sample (e.g., as determined by the machine learning algorithm applied by the processing circuitry). In such examples, the processing circuitry may be configured to increase a duration of centrifugation (e.g., repeat centrifugation or “re-spin”) of a particular sample in response to determining that the respective viability score for the particular sample does not satisfy the viability condition because the degree of region separation of the particular sample is low.

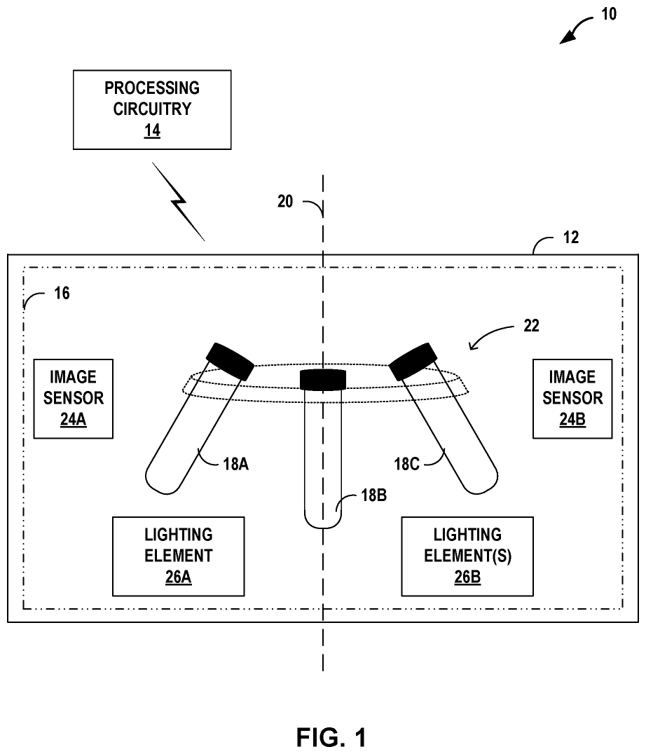

FIG. 1 is a conceptual diagram illustrating an example system 10 in accordance with one or more aspects of this disclosure. In the example of FIG. 1, system 10 includes a centrifuge 12 and processing circuitry 14. In some examples, processing circuitry 14 may be an integrated component of centrifuge 12. In other examples, processing circuitry 14 may be an integrated component of a computing system that includes one or more computing devices. Examples of computing devices may include server devices, personal computers, handheld computers, intermediate network devices, data storage devices, etc. In implementations where the computing system includes two or more computing devices, the computing devices may be geographically distributed or concentrated together (e.g., in a single data center).

Centrifuge 12 may be configured to rotate a set of samples within a chamber 16 defined by an inner surface of centrifuge 12. In the example of FIG. 1, the set of samples may include one or more samples, such as samples18A-18C (collectively, “samples 18”). In general, samples 18 may be cylindrical in shape and about 10 to 15 centimeters (cm) long, although other shapes and sizes of samples 18 are of course possible. Rotation of samples 18 within centrifuge 12 may cause separation of various components of samples 18 based on the densities of those components. For instance, in cases where samples 18 are blood samples, centrifugation of samples 18 may cause the contents of samples 18 to separate into regions of erythrocytes, leukocytes and platelets, and plasma.

Centrifuge 12 may be configured to rotate samples 18 about a central axis 20. In some examples, centrifuge 12 may rotate samples 18 at a predetermined speed and for a predetermined duration. Because centrifuge 12 may rotate samples 18 at a high speed, an uneven distribution of weight of samples 18 relative to central axis 20 may cause centrifuge 12 to vibrate, shake, and even explode. To prevent or reduce the severity of such issues, centrifuge 12 may be configured to automatically decelerate or abort rotation of samples 18 in response to detecting excessive vibration, an excessively unbalanced load, etc.

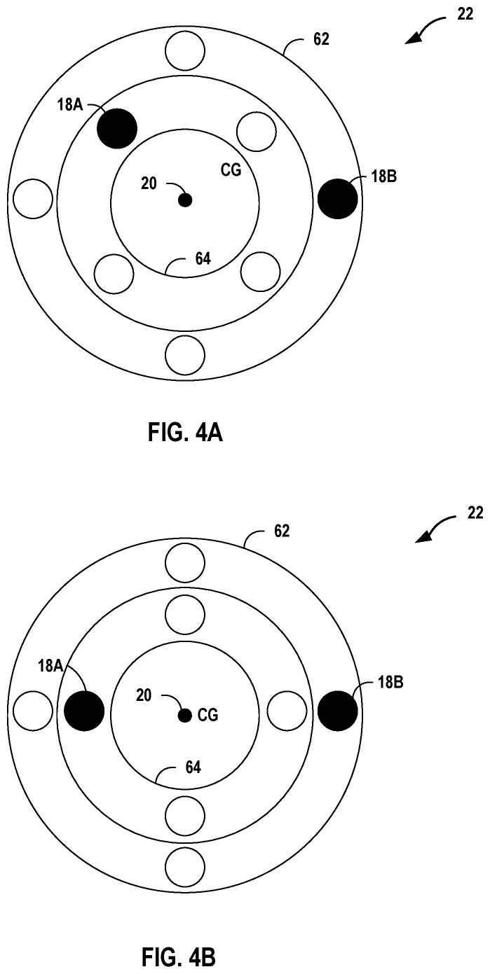

Centrifuge 12 may include a retention assembly 22 configured to retain samples 18. The longitudinal axis of retention assembly 22 may be coaxial with central axis 20. Retention assembly 22 may include one or more rings (shown in, e.g., FIGS. 4A-4B), such as inner ring and an outer ring, configured to retain samples 18. In some cases, retention assembly 22 (e.g., the rings of retention assembly) may define a plurality of recesses configured to receive at least a portion of samples 18. The recesses may be cylindrical, tapered, or otherwise conform to the shape and size of at least a portion of samples 18. Additionally or alternatively, one or more components of retention assembly 22 may be configured to clamp onto samples 18, creating an interference fit that resists movement of samples 18 relative to retention assembly 22 during centrifugation.

As noted above, as part of preparing samples 18 for transportation, analysis, etc., samples 18 may be centrifuged in centrifuge 12. Unfortunately, several issues may occur during centrifugation that in turn affect the viability of one or more of samples 18 for their intended use. For example, the content of sample 18 may render sample 18 not viable for its intended use, irrespective of the amount of centrifugation performed. In another example, centrifuge 12 may spin samples 18 for a predetermined duration, but the predetermined duration may be insufficient such that samples 18 are still not properly separated. In yet another example, centrifuge 12 may abort centrifugation prior to samples 18 properly separating because centrifuge 12 is unbalanced. In yet another example, a lab technician may just accidentally skip “spinning down” one or more of samples 18 in centrifuge 12.

In accordance with techniques of this disclosure, system 10 may be configured to address one or more of these issues. As shown in FIG. 1, centrifuge 12 includes one or more image sensors, such as image sensors 24A-24B (collectively, “image sensors 24”). Image sensors 24 may be configured to generate one or more sets of image data representative of samples 18. In some examples, image sensors 24 may be configured to generate image data representative of samples 18 before, during, and/or after centrifugation of samples 18. Image sensors 24 may be electronic image sensors configured to detect and transmit information used to create an image. For example, image sensors 24 may convert infrared waves, visible light waves, etc., into digital signals. In examples where image sensors 24 are configured to detect visible light waves, centrifuge 12 may include one or more lighting elements 26, such as lighting elements 26A-26B (collectively, “lighting elements 26”) configured to emit light within chamber 16. Lighting elements 26 may be configured to emit light within the chamber at specific times (e.g., at a predetermined frequency), and image sensors 24 may be configured to detect the emitted light at the specific times. Synchronization of the operations of image sensors 24 and lighting elements 26 in this manner may prevent or reduce the flicker effect, which may otherwise impair the quality of image data generated by image sensors 24.

Image sensors 24 may be positioned within chamber 16 to properly generate image data representative of samples 18. Image sensors 24 may be, include, or be a part of one or more image cameras. In some instances, image sensors 24 about 50 millimeters (mm) to 100 mm in order to better capture the pictorial details of samples 18 when positioned within centrifuge 12. In one example, image sensors 24 may be affixed to or otherwise integrated into the side walls defining chamber 16, and positioned at, above or about the same height as samples 18. In another example, image sensors 24 may be affixed to or otherwise integrated into a portion of retention assembly 22, such as rings of retention assembly 22 or a spindle (not shown for ease of illustration) extending through the center of retention assembly 22. In any case, image sensors 24 may be angled such that image sensors 24 generally face the side of samples 18 and thus may properly receive information about the contents of samples 18.

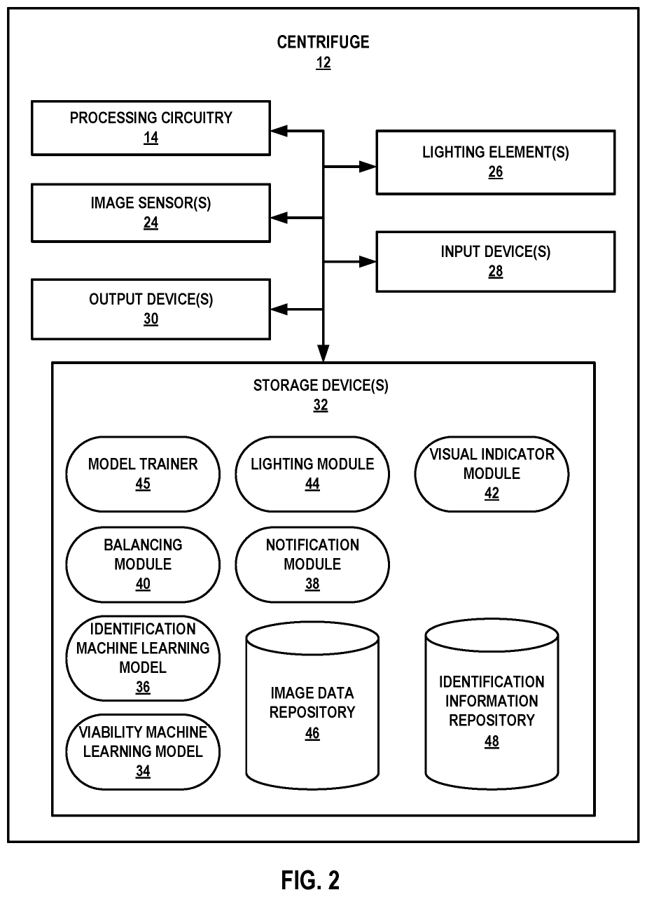

Processing circuitry 14 may be configured to initiate centrifugation of samples 18. During centrifugation of samples 18, image sensors 24 may generate a set of image data. In some cases, the generation of the set of image data may begin when the centrifugation begins (e.g., when samples 18 begin to rotate). Processing circuitry 14 may obtain the set of image data and apply a machine learning model (“ML model”) to the set of image data to determine the viability of each of samples 18. In other words, and as described in greater detail with respect to FIG. 2, the ML model may obtain the set of image data as input and output (e.g., generate) a viability score for each respective sample of the set of samples 18 based on image data representative of the respective sample in the set of image data. The viability score may be indicative of a likelihood of the respective sample being viable for an intended use (e.g., testing). The viability score generated by the ML model may be a numerical value. In some examples, processing circuitry 14 may output a classification, recommendation, etc., based on the numerical value of the viability score. Additionally or alternatively, processing circuitry 14 may perform a task (e.g., increasing a duration of centrifugation) based on the numerical value of the viability score.

The viability score generated by the ML model may be based on various properties (e.g., colors, shapes, textures, volumes, formation of separate regions, volume of each region, etc.) of samples 18. In some examples, the ML model may determine (e.g., identify, classify, predict, etc.) each of the various properties and use the determinations as input for generating the viability score. As one example, the ML model may analyze the formation of separate regions of sample 18A (e.g., as represented by the image data of sample 18A). In general, the ML model may distinguish the regions based on various factors, such as color, texture, and volume. For example, the erythrocytes region of a blood sample is typically a shade of red (e.g., dark red, light red, etc.), opaque, and about 45% of the content volume; the leukocytes and platelets region of a blood sample is typically transparent and about 1% of the content volume; and the plasma region of a blood sample is typically a shade of yellow, translucent, and about 55% of the content volume. As such, in some examples, the ML model may identify areas of extreme contrast in the image data representative of samples 18. The ML model may determine the percentage volume of each region of samples 18 based on the volume of each region of samples 18. When determining the percentage volume of each region of samples 18, the ML model may determine the total volume of samples 18 by subtracting the volume of air from the container size capacity (e.g., container volume) of the respective containers of samples 18.

Thus, based on these various factors, the ML model may determine whether the content of each of samples 18 has formed separate regions of erythrocytes, leukocytes and platelets, and plasma. In some implementations, the ML model may perform binary classification or multiclass classification based on the image data. In binary classification, the output data may include a classification of the input data into one of two different classes. In multiclass classification, the output data may include a classification of the input data into one (or more) of more than two classes. The classifications may be single label or multi-label. The ML model may perform discrete categorical classification in which the input data is simply classified into one or more classes or categories.

As an example, if the ML model determines that the content of sample 18A has formed separate regions, the ML model may assign sample 18A to a first class (e.g., a “separate regions class”); on the other hand, if the ML model determines that the content of sample 18A has not formed separate regions, the ML model may assign sample 18A to a second class (e.g., a “non-separate regions class”). In other examples, the ML model may determine the degree of region separation.

In some implementations, the ML model may perform classification in which the ML model provides, for each of one or more classes, a numerical value descriptive of a degree to which it is believed that the input data should be classified into the corresponding class. The numerical values provided by the ML model may be referred to as “confidence scores” that are indicative of a respective confidence associated with classification of the input into the respective class. In other words, each confidence score may represent a likelihood probability for a classification such that if the confidence score for a particular classification meets or exceeds a probability threshold, the particular classification may be the most probable classification of the input data. In some examples, the confidence scores may be compared to one or more thresholds to render a discrete categorical prediction. The thresholds may be adjustable by trained personnel. In some implementations, only a certain number of classes (e.g., one) with the relatively largest confidence scores may be selected to render a discrete categorical prediction.

As an example, the ML model may render a classification only if the confidence score for the classification is equal to or greater than a threshold value. The ML model may generate a confidence score from 0 to 1 representative of the degree to which it is believed that the content of sample 18A has formed separate regions, where a confidence score of 0 may be associated with a lowest confidence in the classification, and a confidence score of 1 may be associated with a highest confidence in the classification. In this example, the threshold value for a classification that the content of sample 18A has formed separate regions may be 0.8. Thus, if the confidence score for the classification that the content of sample 18A has formed separate regions is equal to or greater than 0.8 (e.g., a confidence score of 0.85), the ML model may render that classification. Otherwise, the ML model may render another classification for which the ML model generated a respective confidence score equal to or greater than the threshold value.

It should be understood that other ranges of confidence scores (e.g., 0 to 100, 0 to −10, etc.), relationships between confidence scores and amount of confidence (e.g., a confidence score of 0 being associated with a highest confidence and a confidence score of 1 being associated with a lowest confidence), threshold values, relationships between confidence scores and threshold values, and/or the like are contemplated by this disclosure.

In a similar manner, the ML model may classify each of the various properties. For instance, the ML model may classify the color of each of the regions of sample 18B. As an example, the ML model may analyze the color (and the shade thereof) of the plasma and in turn classify sample 18B as normal, icteric, hemolytic, or lipemic (thereby indicating whether sample 18B is viable for a particular type of test, such as spectrophotometric analysis). Additionally or alternatively, the ML model may analyze the shade of red of the erythrocytes region of sample 18B and predict a severity of anemia of the patient associated with sample 18B. For instance, based on the shade of red of the erythrocytes region (e.g., in accordance with the World Health Organization (WHO) Hemoglobin Color Scale), the ML model may classify a patient as not anemic (e.g., 12 grams of hemoglobin per deciliter (g/dl) or more), experiencing mild to moderate anemia (e.g., 8-11 g/dl), experiencing marked anemia (e.g., 6-7 g/dl), experiencing severe anemia (e.g., 4-5 g/dl), or experiencing critical anemia (e.g., less than 4 g/dl). As described above, the ML model may generate a confidence score in the classification and compare the confidence score to a threshold value to render the discrete categorical prediction.

The ML model may use the classifications for the various properties of samples 18 as input to generate respective viability scores for samples 18. In some examples, the viability scores may represent a F-score, such as a F1 score. In statistical analysis of binary classification, a F-score or F-measure may be a measure of a test's accuracy. In some examples, the ML model may generate a viability score from 0 to 1, where 0 is associated with a lowest likelihood of viability for an intended use, and 1 is associated with a highest likelihood of viability. It should be understood that other ranges of viability scores are contemplated by this disclosure.

Processing circuitry 14 may determine, for each of samples 18, whether the viability score for the sample satisfies a viability condition. In some examples, processing circuitry 14 may determine that the viability score for the sample satisfies the viability condition when the viability score is equal to or greater than a threshold value, such as 0.9. Thus, if the ML model generates a viability score of 0.95 for sample 18C, and if the threshold value is 0.9, then processing circuitry 14 may determine that the viability score for sample 18C satisfies the viability condition. On the other hand, if the ML model generates a viability score of 0.6 for sample 18C, then processing circuitry 14 may determine that the viability score for sample 18C does not satisfy the viability condition. In any case, processing circuitry 14 may output, for each of samples 18, whether the respective viability score satisfies the viability condition.

In examples where the viability score for a sample is based, at least in part, on a degree of region separation of the sample, processing circuitry 14 may be configured to increase a duration of centrifugation (e.g., repeat centrifugation or “re-spin”) of a particular sample in response to determining that the respective viability score for the particular sample does not satisfy the viability condition because the degree of region separation of the particular sample is low. In other words, if the viability score for the particular sample would satisfy the viability condition but for a low degree of region separation, processing circuitry 14 may be configured to increase the duration of centrifugation (e.g., repeat centrifugation or “re-spin”) of the particular sample.

For example, if the ML model generates a viability score of 0.89 for sample 18B, and if the threshold value is 0.9, then processing circuitry 14 may determine that the viability score for sample 18B does not satisfy the viability condition. However, if the degree of region separation of sample 18B is low (or otherwise capable of being increased), then the ML model may generate a viability score greater than 0.89 (e.g., 0.9 or more) for sample 18B if the degree of region separation of sample 18B increased, which can occur by increasing a duration of centrifugation of sample 18B. Accordingly, processing circuitry 14 may “re-spin” sample 18B, and the ML model may determine the viability score of sample 18B after the re-spin.

In some examples, the ML model may classify a particular sample as not being capable of separating (e.g., increasing duration of centrifugation may not increase the degree of region separation). In such examples, processing circuitry 14 may output a notification indicating whether the viability score for the particular sample satisfies the viability condition, and if the viability score does not satisfy the particular sample, processing circuitry 14 may not increase the duration of centrifugation of the particular sample.

Accordingly, these techniques may enable advanced analysis for determining the viability of samples 18 that constitutes an improvement upon the existing art. For instance, the techniques described herein may exceed the abilities of the human eye to detect colors, shapes, and separation. The collected clinical data can be further evaluated via the clinical studies to determine solutions for other lab related problems. The cost of undetected flaws in lab specimens may be greatly reduced and can be resolved at a doctor's office before involving costly lab personnel.

FIG. 2 is a block diagram illustrating example components of centrifuge 12 in accordance with one or more aspects of this disclosure. FIG. 2 illustrates only one example of centrifuge 12, without limitation on many other example configurations of centrifuge 12. As shown in the example of FIG. 2, centrifuge 12 includes processing circuitry 14, image sensors 24, lighting elements 26, one or more input devices 28, one or more output devices 30, and one or more storage devices 32. Centrifuge 12 may include other components.

Processing circuitry 14 may include one or more processors configured to perform processing functions. For instance, one or more of processors may be a microprocessor, application-specific integrated circuit (ASIC), field-programmable gate array (FPGA), or other type of processing circuit. In some examples, processing circuitry 14 may read and execute instructions stored by storage devices 32. Processing circuitry 14 may include fixed-function processors and/or programmable processors.

Although shown in FIG. 2 as being integrated into centrifuge 12, processing circuitry 14 may be a separate component integrated into another device or system, such as a personal computer. Thus, it should be understood that techniques of this disclosure may be performed, at least in part, by processing circuitry not integrated into centrifuge 12 and that the examples described herein are for purposes of illustration and are without limitation.

Centrifuge 12 may be configured to receive input from a user via input devices 28. Examples of input devices 28 may include buttons and other user interface elements that a user may actuate to provide input. Additional examples of input devices 28 may include a presence-sensitive display, a mouse, a keyboard, a voice responsive system, video camera, microphone or any other type of device for detecting a command from a user.

Centrifuge 12 may include output devices 30. Output devices 30 may be configured to provide output to a user using tactile, audio, or video stimuli. Examples of output devices may include a presence-sensitive display, a sound card, a video graphics adapter card, or any other type of device for converting a signal into an appropriate form understandable to humans or machines. Additional examples of output devices 30 may include a speaker, a cathode ray tube (CRT) monitor, a liquid crystal display (LCD), or any other type of device that may generate intelligible output to a user.

Centrifuge 12 may include storage devices 32. Storage devices 32 may include one or more computer-readable storage media. For example, storage devices 32 may be configured for long-term, as well as short-term storage of information, such as instructions, data, or other information used by centrifuge 12. In some examples, storage devices 32 may include non-volatile storage elements. Examples of such non-volatile storage elements include magnetic hard discs, optical discs, solid state discs, and/or the like. In other examples, in place of, or in addition to the non-volatile storage elements, storage devices 32 may include one or more so-called “temporary” memory devices, meaning that a primary purpose of these devices may not be long-term data storage. For example, the devices may comprise volatile memory devices, meaning that the devices may not maintain stored contents when the devices are not receiving power. Examples of volatile memory devices include random-access memories (RAM), dynamic random-access memories (DRAM), static random-access memories (SRAM), etc.

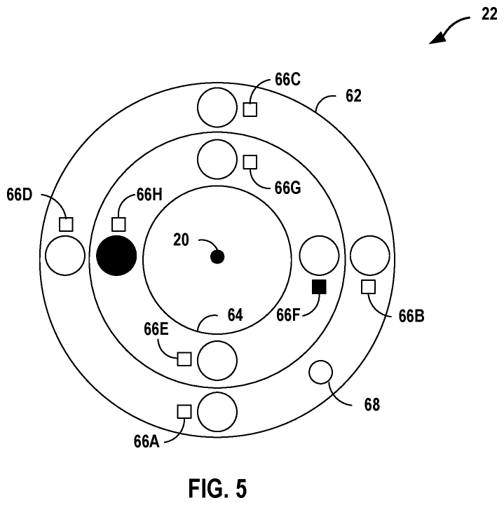

As shown in FIG. 2, storage devices 32 includes a viability machine learning model 34, an identification machine learning model 36, a notification module 38, a balancing module 40 (discussed in greater detail with respect to FIGS. 4A-B), a visual indicator module 42 (discussed in greater detail with respect to FIG. 5), a lighting module 44 (discussed in greater detail with respect to FIG. 5), a model trainer 45, an image data repository 46, and an identification information repository 48. Image data generated (e.g., before, during, and/or after a cycle of centrifugation) by image sensors 24 may be stored in image data repository 46. Identification information associated with samples 18 may be stored in identification information repository 48.

Viability machine learning model 34 and identification machine learning model 36 may each include one or more of various different types of ML models that perform classification, regression, clustering, anomaly detection, recommendation generation, and/or other tasks. For instance, viability machine learning model 34 and identification machine learning model 36 may each be or include one or more artificial neural networks (“neural networks”). A neural network may include a group of connected nodes, which also may be referred to as neurons or perceptrons. A neural network may be organized into one or more layers. Neural networks that include multiple layers may be referred to as “deep” networks. A deep network may include an input layer, an output layer, and one or more hidden layers positioned between the input layer and the output layer. The nodes of the neural network may be connected or non-fully connected.

In some implementations, the neural networks may be feed forward neural networks. In feed forward networks, the connections between nodes do not form a cycle. For example, each connection may connect a node from an earlier layer to a node from a later layer. In some instances, the neural networks may be recurrent neural networks. In recurrent neural networks, at least some of the nodes may form a cycle. Recurrent neural networks may be especially useful for processing input data that is sequential in nature. In particular, in some instances, a recurrent neural network may pass or retain information from a previous portion of the input data sequence to a subsequent portion of the input data sequence through the use of recurrent or directed cyclical node connections. In some examples, the input data sequence may include time-series data (e.g., sensor data versus time or imagery captured at different times). For example, a recurrent neural network may analyze image data representative of samples 18 versus time to detect or predict the effect of the duration of centrifugation on various properties of samples 18, including the degree of region separation.

In some implementations, the neural networks may be convolutional neural networks. A convolutional neural network may include one or more convolutional layers that perform convolutions over input data using learned filters or kernels. Convolutional neural networks may be especially useful for vision problems such as when the input data includes imagery such as still images or video.

As noted above, viability machine learning model 34 and identification machine learning model 36 may each perform classification in which viability machine learning model 34 and identification machine learning model 36 provide, for each of one or more classes, a numerical value descriptive of a degree to which it is believed that the input data should be classified into the corresponding class. Additionally or alternatively, viability machine learning model 34 and identification machine learning model 36 may each output a probabilistic classification. For example, viability machine learning model 34 and identification machine learning model 36 may may predict, given a sample input, a probability distribution over a set of classes. Thus, rather than outputting only the most likely class to which the sample input should belong, viability machine learning model 34 and identification machine learning model 36 may output, for each class, a probability that the sample input belongs to such class. In some implementations, the probability distribution over all possible classes may sum to one. In some implementations, a Softmax function, or other type of function or layer may be used to squash a set of real values respectively associated with the possible classes to a set of real values in the range (0, 1) that sum to one.

In some examples, the probabilities provided by the probability distribution may be compared to one or more thresholds to render a discrete categorical prediction. In some implementations, only a certain number of classes (e.g., one) with the relatively largest predicted probability may be selected to render a discrete categorical prediction.

In cases in which viability machine learning model 34 and identification machine learning model 36 perform classification, viability machine learning model 34 and identification machine learning model 36 may be trained using supervised learning techniques. For example, viability machine learning model 34 and identification machine learning model 36 may be trained on a training dataset that includes training examples labeled as belonging (or not belonging) to one or more classes.

In some implementations, viability machine learning model 34 and identification machine learning model 36 may perform regression to provide output data in the form of a continuous numeric value. The continuous numeric value may correspond to any number of different metrics or numeric representations, including, for example, densities, scores, or other numeric representations. As examples, viability machine learning model 34 and identification machine learning model 36 may perform linear regression, polynomial regression, or nonlinear regression. As examples, viability machine learning model 34 and identification machine learning model 36 may perform simple regression or multiple regression. As described above, in some implementations, a Softmax function or other function or layer may be used to squash a set of real values respectively associated with a two or more possible classes to a set of real values in the range (0, 1) that sum to one.

Viability machine learning model 34 and identification machine learning model 36 may be trained in a variety of ways. For example, viability machine learning model 34 and identification machine learning model 36 may be trained at a training computing system then provided for storage and/or implementation at one or more devices, such as centrifuge 12. In another example, a model trainer configured to train viability machine learning model 34 and identification machine learning model 36 may execute locally at centrifuge 12.

In some implementations, a model trainer 45 may train viability machine learning model 34 and identification machine learning model 36 in an offline fashion or an online fashion. In offline training (also known as batch learning), model trainer 45 may train viability machine learning model 34 and identification machine learning model 36 on the entirety of a static set of training data. In online learning, model trainer 45 may continuously train (or re-train) viability machine learning model 34 and identification machine learning model 36 as new training data becomes available (e.g., while viability machine learning model 34 and identification machine learning model 36 are used to perform inference). In some implementations, model trainer 45 may use decentralized training techniques such as distributed training, federated learning, or the like to train, update, or personalize viability machine learning model 34 and identification machine learning model 36.

In some implementations, model trainer 45 may train viability machine learning model 34 and identification machine learning model 36 using supervised learning, in which model trainer 45 trains viability machine learning model 34 and identification machine learning model 36 on a training dataset that includes instances or examples that have labels. The labels may be manually applied by experts, generated through crowd-sourcing, or provided by other techniques (e.g., by physics-based or complex mathematical models).

In some implementations, model trainer 45 may train viability machine learning model 34 and identification machine learning model 36 by optimizing an objective function. For example, in some implementations, the objective function may be or include a loss function that compares (e.g., determines a difference between) output data generated by the model from the training data and labels (e.g., ground-truth labels) associated with the training data. For example, the loss function may evaluate a sum or mean of squared differences between the output data and the labels. In some examples, the objective function may be or include a cost function that describes a cost of a certain outcome or output data. Other examples of the objective function may include margin-based techniques such as, for example, triplet loss or maximum-margin training.

Model trainer 45 may perform one or more of various optimization techniques to optimize the objective function. For example, the optimization technique(s) may minimize or maximize the objective function. Example optimization techniques include Hessian-based techniques and gradient-based techniques, such as, for example, coordinate descent; gradient descent (e.g., stochastic gradient descent); subgradient methods; etc. Other optimization techniques include black box optimization techniques and heuristics.

In some implementations, model trainer 45 may use backward propagation of errors in conjunction with an optimization technique (e.g., gradient based techniques) to train viability machine learning model 34 and identification machine learning model 36 (e.g., when machine-learned model is a multi-layer model such as an artificial neural network). For example, model trainer 45 may perform an iterative cycle of propagation and model parameter (e.g., weights) update to train viability machine learning model 34 and identification machine learning model 36. Example backpropagation techniques include truncated backpropagation through time, Levenberg-Marquardt backpropagation, etc.

In some implementations, model trainer 45 may train viability machine learning model 34 and identification machine learning model 36 using unsupervised learning techniques. Unsupervised learning may include inferring a function to describe hidden structure from unlabeled data. For example, a classification or categorization may not be included in the data. Model trainer 45 may use unsupervised learning techniques to produce machine-learned models capable of performing clustering, anomaly detection, learning latent variable models, or other tasks.

Model trainer 45 may train viability machine learning model 34 and identification machine learning model 36 using semi-supervised techniques which combine aspects of supervised learning and unsupervised learning. Model trainer 45 may train or otherwise generate viability machine learning model 34 and identification machine learning model 36 using evolutionary techniques or genetic algorithms. In some implementations, model trainer 45 may train viability machine learning model 34 and identification machine learning model 36 using reinforcement learning.

In some implementations, model trainer 45 may perform one or more generalization techniques during training to improve the generalization of viability machine learning model 34 and identification machine learning model 36. Generalization techniques may help reduce overfitting of viability machine learning model 34 and identification machine learning model 36 to the training data. Example generalization techniques include dropout techniques; weight decay techniques; batch normalization; early stopping; subset selection; stepwise selection; etc.

Viability machine learning model 34 may be similar, if not substantially similar, to the ML model described with respect to FIG. 1. For instance, viability machine learning model 34 may, based on image data stored in image data repository 46, determine various properties (e.g., colors, shapes, textures, formation of separate regions, volume of each region, etc.) of samples 18 and, based on those determined properties, generate a viability score. In some examples, determining the various properties of samples 18 may include performing spectrometry, polarimetry, etc. Spectrometry is a technique that may be used to determine the detailed compositions of a fluid. Polarimetry is a technique that may be used to measure a refractive index of a fluid, which in turn may be used to determine shapes, regions, and colors of the fluid. Implementation of such techniques, which are typically far more precise and accurate than human observation, may add significant scientific data to corroborate a determination of sample viability.

The viability score generated by viability machine learning model 34 may be based on one or more of the colors, shapes, textures, formation of separate regions, volume of each region, etc., as determined by viability machine learning model 34. In some examples, the viability score may be an average of the determinations (e.g., classifications, predictions, etc.) for the various properties of samples 18 by viability machine learning model 34.

Processing circuitry 14 may output (e.g., via output devices 30) the viability scores generated by viability machine learning model 34 and whether the viability scores satisfy the viability condition. For example, processing circuitry 14 may execute a notification module 38 to output a notification (e.g., a communication displayed via output devices 30 or transmitted to a personal computing device of a user) of which of samples 18 are viable because they have viability scores that satisfy the viability condition, which of samples 18 are not viable because they have viability scores that do not satisfy the viability condition, which of samples 18 are not viable but likely would be if the duration of centrifugation were increased because they have viability scores that would satisfy the viability condition if the degree of region separation were higher, etc.

In some examples, processing circuitry 14 may control centrifuge 12 to increase the duration of centrifugation (e.g., by repeating a cycle of centrifugation) for the samples 18 that are not viable but likely would be if the duration of centrifugation were increased. In such examples, notification module 38 may output a notification that the duration of centrifugation was increased. Additionally or alternatively, notification module 38 may output a notification of errors and/or issues that occurred during centrifugation, such as whether centrifugation of samples 18 was prematurely terminated because of centrifuge 12 being unbalanced, whether one or more of samples 18 were misidentified, etc.

Before centrifugation of samples 18, image sensors 24 may generate a set of image data representative of samples 18. Processing circuitry 14 may obtain the set of image data and apply a machine learning model, such as identification machine learning model 36, to confirm whether the identification information associated with samples 18 is consistent with the content of samples 18. For example, identification machine learning model 36 may determine that the content of one of samples 18 is empty in accordance with techniques of this disclosure even though the identification information associated with that sample indicates that the content is supposed to be a blood sample, meaning that the identification information is inaccurate.

Confirming the identification information associated with samples 18 in accordance with techniques of this disclosure may help identify samples that are mislabeled, which otherwise could invalidate the results of testing. In some examples, processing circuitry 14 may abort initiation of centrifugation of samples 18 or prematurely terminate centrifugation of the samples in response to processing circuitry 14 determining that one or more of the confirmation scores do not satisfy the confirmation condition. This may be advantageous because the appropriate speed and duration of centrifugation for a sample may depend on the contents of the sample, in turn making correctly identifying samples 18 crucial.

Processing circuitry 14 may obtain the identification information from a respective identification element associated with (e.g., on the container of) each of samples 18. The identification element may be a so-called “smart cap” that may be read using radio-frequency identification (RFID) or some other element, such as stickers on the containers of samples 18, configured to communicate information. In some examples, identification machine learning model 36 may be configured to generate, for each of samples 18, a confirmation score for the respective sample, where the confirmation score is indicative of a likelihood of the identification information associated with the respective sample being accurate.

Identification machine learning model 36 may use as input the image data generated by image sensors 24 before centrifugation and identification information stored in identification information repository 48 to output a confirmation score for each respective sample of the set of samples 18. The identification information may include, but is not limited to, a sample identification number, sample properties (e.g., sample weight, intended use, etc.), container size capacity, and patient data (e.g., patient name, race, age, ethnicity, regional demographics (e.g., related to food supplies and water conditions), species in veterinary applications, etc.).

Like viability machine learning model 34, identification machine learning model 36 may perform classification in which identification machine learning model 36 provides “confidence scores” associated with classification of various properties for identifying samples 18, such as color, volume, weight, container size capacity, etc. In some examples, the confidence scores may be compared to one or more thresholds (e.g., 0.8). The thresholds may be adjustable by trained personnel. If the confidence scores satisfy the thresholds, identification machine learning model 36 may render the classifications. For instance, if sample 18A is empty but the identifying information indicates that sample 18A is supposed to contain a blood sample, identification machine learning model 36 may, based on the image data representative of sample 18A, generate a confidence score of 0.9 in the classification that sample 18A is empty. If the threshold for such a classification is 0.8, then because the confidence score of 0.9 is greater than the threshold of 0.8, identification machine learning model 36 may render the classification that sample 18A is empty and use that classification in determining the confirmation score for sample 18A.

Processing circuitry 14 may compare the confirmation scores (which may be numerical values) generated by identification machine learning model 36 to a confirmation condition to determine whether the confirmation scores satisfy the confirmation condition. In some examples, processing circuitry 14 may determine that the confirmation score for the sample satisfies the confirmation condition when the confirmation score is equal to or greater than a threshold value, such as 0.8. Thus, if identification machine learning model 36 generates a confirmation score of 0.85 for sample 18A, and if the threshold value is 0.8, then processing circuitry 14 may determine that the confirmation score for sample 18A satisfies the confirmation condition. On the other hand, if identification machine learning model 36 generates a confirmation score of 0.7 for sample 18A, then processing circuitry 14 may determine that the confirmation score for sample 18A does not satisfy the confirmation condition.

In any case, notification module 38 may output, for each of samples 18, a notification of whether the respective confirmation score satisfies the confirmation condition. In this way, notification module 38 may indicate which of samples 18 are correctly identified and which are not. In some examples, processing circuitry 14 may abort initiation of centrifugation of samples 18 or prematurely terminate a cycle of centrifugation if processing circuitry 14 determines that the one or more of the confirmation scores do not satisfy the confirmation condition. Furthermore, notification module 38 may output notifications indicating which of samples 18 completed centrifugation and/or which of samples 18 did not complete centrifugation, thereby potentially reducing a likelihood of a user accidentally skipping “spinning down” of any of samples 18.

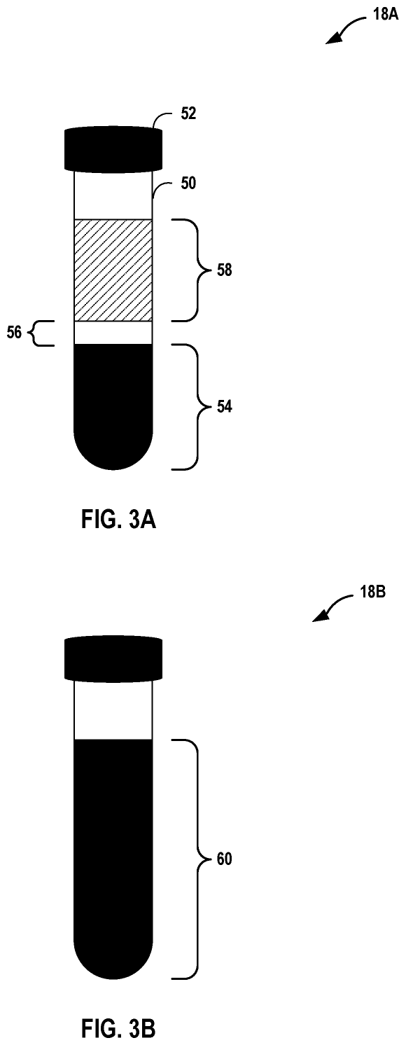

FIGS. 3A-B are conceptual diagrams illustrating example samples in accordance with one or more aspects of this disclosure. Samples 18 may be stored inside containers (e.g., test tubes) with an integrated (or attached) identification element. For instance, as shown in FIG. 3A, a container 50 includes sample 18A. A respective identification element 52 may be integrated into a portion of each container 50, such as a lid of container 50. The lid of container 50 may be a so-called “smart cap.” Identification element 52 may communicate identification information that processing circuitry 14 stores in identification information repository 48. For example, centrifuge 12 may include a RFID reader that reads the smart caps of samples 18 and stores the identification information in identification information repository 48. In the example of FIG. 3A, sample 18A is a blood sample. During (as well as after) centrifugation of sample 18A, image sensors 24 may generate image data representative of sample 18A. In other words, the image data representative of sample 18A during centrifugation may appear similar, if not substantially similar, to the illustration of FIG. 3A.

Sample 18A may be centrifuged, and the content of sample 18A may form separate regions. As shown in FIG. 3A, the separate regions include an erythrocytes region 54, a leukocytes and platelets region 56, and a plasma region 58. Erythrocytes region 54, leukocytes and platelets region 56, and plasma region 58 may differ from each other in terms of color, volume, etc., as described above. Viability machine learning model 34 may analyze the image data representative of sample 18A to generate the viability score. For example, Viability machine learning model 34 may determine that sample 18A exhibits a high degree of region separation and normal colors, shapes, and volumes for the respective regions. Accordingly, Viability machine learning model 34 may generate a viability score for sample 18A that satisfies the viability condition, indicating a high likelihood of viability.

In the example of FIG. 3B, sample 18B is a blood sample. During (as well as after) centrifugation of sample 18B, image sensors 24 may generate image data representative of sample 18B. In other words, the image data representative of sample 18B during centrifugation may appear similar, if not substantially similar, to the illustration of FIG. 3B. Like sample 18A, sample 18B may be stored inside a container with an integrated identification element. Furthermore, like sample 18A, sample 18B may be centrifuged. However, as shown in FIG. 3B, sample 18B may only have a region 60. Viability machine learning model 34 may analyze the image data representative of sample 18B to generate the viability score. For example, Viability machine learning model 34 may determine that sample 18B exhibits a low degree of region separation and that various regions are missing. Accordingly, Viability machine learning model 34 may generate a viability score for sample 18A that does not satisfy the viability condition, indicating a low likelihood of viability.

FIGS. 4A-B are conceptual diagrams illustrating an example of retention assembly 22 in accordance with one or more aspects of this disclosure. In some examples, retention assembly 22 may be a circular-ring type holding system that is moveable within centrifuge 12 by one or more actuators. The actuators may reposition samples 18 in a more equal manner to more evenly distribute the weight of samples 18. As shown in FIGS. 4A-B, retention assembly 22 includes an outer ring 62 and an inner ring 64. Actuators may be configured to move inner ring 64 and outer ring 62 relative to central axis 20, which may extend through a center of retention assembly 22. Inner ring 64 and outer ring 62 may each be configured to retain one or more of samples 18, such as sample 18A, sample 18B, etc. For example, inner ring 64 and outer rings 62 may define one or more recesses configured to receive samples 18. In some examples, centrifuge 12 may include magnetic sensors, optical sensors (e.g., image sensors 24), and/or switches to determine which recesses samples 18 occupy. One or more of these sensors may be integrated into retention assembly 22.

A set of samples 18 has a center of gravity (CG). Depending on the positioning of samples 18, the CG of samples 18 may be closer or further away from central axis. For example, the positioning of samples 18 shown in FIG. 4A results in a CG further away from central axis 20 than the positioning of samples 18 shown in FIG. 4B.

When a center of gravity (CG) of samples 18 is not sufficiently close to central axis 20, centrifuge 12 may vibrate, shake, and even explode. Accordingly, processing circuitry 14 may execute balancing module 40 to control retention assembly 22 to adjust CG of samples 18 relative to central axis 20. Balancing module 40 may attempt to minimize the distance between the CG of samples 18 and central axis 20. To do so, balancing module 40 may obtain (e.g., from each respective identification element 52) the respective weights of samples 18 and obtain (e.g., from image sensors 24) the respective positions of samples 18. Balancing module 40 may calculate the CG of samples 18 using the following equations:

$$C {G}_{x} = \frac{{W}_{1} {x}_{1} + {W}_{2} {x}_{2} + … + {W}_{n} {x}_{n}}{W}$$

$$C {G}_{y} = \frac{{W}_{1} {y}_{1} + {W}_{2} {y}_{2} + … + {W}_{n} {y}_{n}}{W}$$

where CGₓ represents the x-coordinate of the CG of samples 18 relative to central axis 20 (which may be the origin), CG_{y} represents the y-coordinate of the CG of samples 18 relative to central axis 20, W₁ represents the weight of a first sample of samples 18, x₁ represents the x-coordinate value of the position of the first sample relative to central axis 20, y₁ represents the y-coordinate value of the position of the first sample relative to central axis 20, W₂ represents the weight of a second sample of samples 18, x₂ represents the x-coordinate value of the position of the second sample relative to central axis 20, y₂ represents the y-coordinate value of the position of the second sample relative to central axis 20, Wₙ represents the weight of a nth sample of samples 18, xₙ represents the x-coordinate value of the position of the nth sample relative to central axis 20, yₙ represents the y-coordinate value of the position of the nth sample relative to central axis 20, and W represents the total weight of samples 18.

Using the equations above, balancing module 40 may control retention assembly 22 to rotate at least one of inner ring 64 or outer ring 62 of retention assembly 22 about central axis 20 to minimize CGₓ and/or CG_{y}. For example, as shown in FIG. 4B, balancing module 40 may control retention assembly 22 to rotate inner ring 64 such that sample 18A is positioned diametrically opposite of sample 18B, thereby decreasing the distance between the CG of samples 18 and central axis 20.

FIG. 5 is a conceptual diagram illustrating an example of retention assembly 22 accordance with one or more aspects of this disclosure. As shown in FIG. 5, retention assembly 22 may include one or more visual indicators, such as visual indicators 66A-66H (collectively, “visual indicators 66”). FIG. 5 illustrates only one example of visual indicators 66, without limitation on many other example configurations of visual indicators 66. Other configurations of visual indicators 66 should be readily apparent to one of ordinary skill in the art.

In some examples, visual indicators 66 are light emitting diodes (LEDs) integrated into retention assembly 22. Alternatively, visual indicators 66 may be markings that output devices 30 may display or otherwise output to a user. Processing circuitry 14 may execute visual indicator module 42 to control visual indicators 66 to indicate a respective position for each of samples 18. For example, as shown in FIG. 5, visual indicator module 42 may cause visual indicator 66F to illuminate, indicating to a user to insert the next sample into the recess adjacent to visual indicator 66F.

Retention assembly 22 may include a marker 68. Marker 68 may be configured to indicate an angular position of retention assembly 22. Processing circuitry 14 may inventory (as well as track) each of samples 18 positioned in centrifuge 12 using marker 68. In some examples, marker 68 may be a magnetic element that centrifuge 12 detects (e.g., whenever marker 68 passes a magnetic sensor, such as a Hall Effect sensor, of centrifuge 12). Accordingly, processing circuitry 14 may determine the positions of samples 18 inserted into retention assembly 22 based on the position of marker 68. In some implementations, image sensors 24 generates image data (e.g., captures an image) of each of samples 18 once per revolution of retention assembly 22, as indicated by marker 68. Image sensors 24 may generate accurate image data at the correct intervals due to the same timing characteristics of retention assembly 22.

Processing circuitry 14 may execute a lighting module 44 to control lighting elements 26 to emit light within chamber 16 at specific times. Lighting module 44 may use the position of marker 68 to determine the specific times to control lighting elements 26 to emit light. This may facilitate the generation of image data when image sensors 24 have a relatively direct view of samples 18 and when there is adequate lighting for the generated image data to be of relatively good quality.

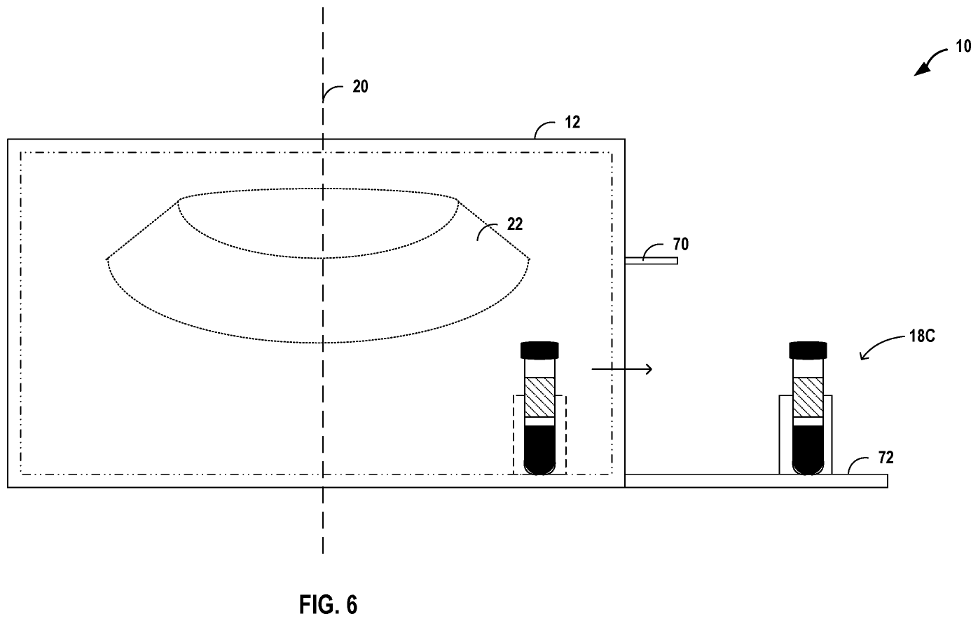

FIG. 6 is a conceptual diagram illustrating an example of an ejection mechanism 70 in accordance with one or more aspects of this disclosure. FIG. 6 illustrates only one example of ejection mechanism 70, without limitation on many other example configurations of ejection mechanism 70. Ejection mechanism 70 may be configured to eject any of samples 18. Ejection mechanism 70 may operate in conjunction with retention assembly 22 to eject samples 18. For example, retention assembly 22 may release a clamp securing any of samples 18 to allow samples 18 to descend (in a controlled fashion) through ejection mechanism 70 and exit centrifuge 12. Centrifuge 12 may come to a stop before ejecting any of samples 18.

Processing circuitry 14 may be configured to control ejection mechanism 70 to eject a particular sample, such as sample 18C, of samples 18 from chamber 16 in response to determining that the respective viability score for the particular sample satisfies the viability condition. In some examples, ejected samples may be positioned proximate a thermal element 72 configured to heat or cool ejected samples to regulate the temperature of the ejected samples.

In some examples, ejection mechanism 70 may not eject samples 18 outside of retention assembly 22. Instead, ejection mechanism 70 may eject one or more of samples 18 to the center of retention assembly 22 to reduce the centrifugal force applied to the ejected samples. Centrifuge 12 may include thermal element 72, and thermal element 72 may be disposed within centrifuge 12 such that ejected samples are positioned proximate to thermal element 72.

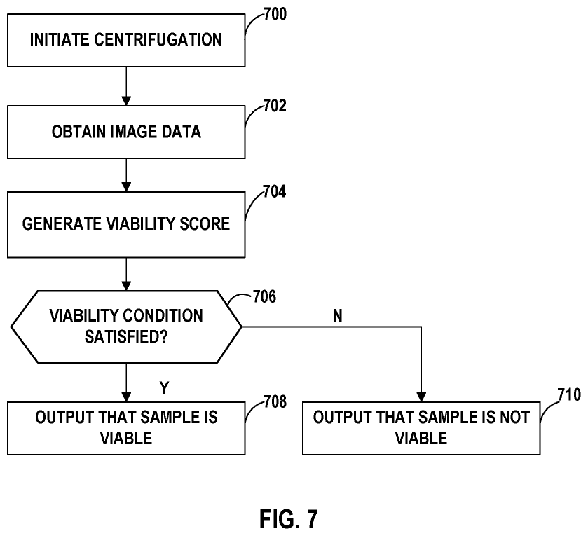

FIG. 7 is a flowchart illustrating an example method in accordance with one or more techniques of this disclosure. Processing circuitry 14 may initiate centrifugation of samples 18 (700). During centrifugation of samples 18, image sensors 24 may generate a set of image data and store the image data in image data repository 46. Processing circuitry 14 may obtain the set of image data from image data repository 46 (702). Processing circuitry 14 may apply viability machine learning model 34 to the set of image data to generate a viability score for each respective sample of the set of samples 18 based on image data representative of the respective sample in the set of image data (704).

Processing circuitry 14 may determine, for each of samples 18, whether the generated viability score for the sample satisfies a viability condition (706). In some examples, processing circuitry 14 may determine that the viability score for the sample satisfies the viability condition when the viability score is equal to or greater than a threshold value, such as 0.9. If processing circuitry 14 determines that the viability score for the sample satisfies the viability condition (Y of 706), notification module 38 may output a notification indicating that the sample is viable (708). If processing circuitry 14 determines that the viability score for the sample does not satisfy the viability condition (N of 706), notification module 38 may output a notification indicating that the sample is not viable (710). In this way, the techniques of this disclosure may enable the identification of which of samples 18 viable and which are not.

This disclosure includes the following examples.

Example 1: A method includes initiating, by processing circuitry, centrifugation of a set of one or more samples within a chamber of a centrifuge and about a central axis; obtaining, by the processing circuitry, a set of image data generated by an image sensor during centrifugation of the set of samples, wherein the set of image data is representative of the set of samples; and for each respective sample of the set of samples: applying, by processing circuitry, a machine learning model configured to generate, based on image data representative of the respective sample in the set of image data, a viability score for the respective sample, wherein the viability score is indicative of a likelihood of the respective sample being viable for an intended use; determining, by the processing circuitry, whether the viability score for the sample satisfies a viability condition; and outputting, by the processing circuitry, a notification indicating whether the viability score for the sample satisfies the viability condition.

Example 2: The method of example 1, wherein the viability score for the sample satisfies the viability condition when the viability score is equal to or greater than a threshold value.

Example 3: The method of any of examples 1 and 2, further including controlling, by the processing circuitry, a retention assembly of the centrifuge to adjust a center of gravity of the set of samples relative to the central axis.

Example 4: The method of example 3, wherein controlling the retention assembly includes rotating, by the processing circuitry, at least one of an inner ring or an outer ring of the retention assembly about the central axis, wherein the inner ring and the outer ring are each configured to retain one or more samples of the set of samples.

Example 5: The method of any of examples 1 through 4, further including controlling, by the processing circuitry, a visual indicator of the centrifuge to indicate a respective position for each sample of the set of samples.

Example 6: The method of any of examples 1 through 5, further includes for each respective sample of the set of samples, obtaining, by the processing circuitry, identification information associated with the respective sample based on a respective identification element associated with the respective sample, wherein the identification information associated with the respective sample includes at least one of a patient name, intended use, or a sample weight.

Example 7: The method of any of examples 1 through 6, wherein the machine learning model is a first machine learning model, wherein the set of image data is a first set of image data, and wherein the method further includes: obtaining, by the processing circuitry, a second set of image data generated by the image sensor before centrifugation of the set of samples, wherein the second set of image data is representative of the set of samples; and for each respective sample of the set of samples: obtaining, by the processing circuitry, identification information associated with the respective sample based on a respective identification element associated with the respective sample; applying, by processing circuitry, a second machine learning model configured to generate, based on image data representative of the respective sample in the second set of image data, a confirmation score for the respective sample, wherein the confirmation score for the respective sample is indicative of a likelihood of the identification information associated with the respective sample being accurate; determining, by the processing circuitry, whether the confirmation score for the respective sample satisfies a confirmation condition; and outputting, by the processing circuitry, a notification indicating whether the confirmation score for the respective sample satisfies the confirmation condition.

Example 8: The method of example 7, further including terminating, by the processing circuitry, centrifugation of the set of samples in response to the processing circuitry determining that one or more of the respective confirmation scores do not satisfy the confirmation condition.

Example 9: The method of any of examples 1 through 8, further including controlling, by the processing circuitry an ejection mechanism of the centrifuge to eject a particular sample of the set of samples from the chamber in response to the processing circuitry determining that the respective viability score for the particular sample satisfies the viability condition.

Example 10: The method of any of examples 8 and 9, wherein the ejection mechanism ejects the particular sample proximate a thermal element configured to heat or cool the particular sample to regulate a temperature of the particular sample.

Example 11: The method of any of examples 1 through 10, wherein, for each respective sample of the set of samples, the viability score for the respective sample is based, at least in part, on a degree of region separation of the respective sample, and wherein the method further includes: increasing a duration of centrifugation of a particular sample of the set of samples in response to the processing circuitry determining that the viability score for the particular sample does not satisfy the viability condition because the degree of region separation of the particular sample is low.

Example 12: A centrifuge includes a chamber configured to contain a set of one or more samples; an image sensor configured to generate image data; and processing circuitry configured to: initiate centrifugation of the set of samples about a central axis; obtain a set of image data generated by the image sensor during centrifugation of the set of samples, wherein the set of image data is representative of the set of samples; and for each respective sample of the set of samples: apply a machine learning model configured to generate, based on image data representative of the respective sample in the set of image data, a viability score for the respective sample, wherein the viability score is indicative of a likelihood of the respective sample being viable for an intended use; determine whether the viability score for the respective sample satisfies a viability condition; and output a notification indicating whether the viability score for the respective sample satisfies the viability condition.

Example 13: The centrifuge of example 12, wherein the centrifuge further includes a retention assembly, and wherein the processing circuitry is further configured to control the retention assembly to adjust a center of gravity of the set of samples relative to the central axis.

Example 14: The centrifuge of any of examples 12 and 13, wherein the centrifuge further includes a visual indicator, and wherein the processing circuitry is further configured to control the visual indicator to indicate a respective position for each sample of the set of samples.

Example 15: The centrifuge of any of examples 12 through 14, wherein the processing circuitry is further configured to, for each respective sample of the set of samples, obtain identification information associated with the respective sample based on a respective identification element associated with the respective sample, wherein the identification information associated with the respective sample includes at least one of a patient name, intended use, or a sample weight.

Example 16: The centrifuge of any of examples 12 through 15, wherein the machine learning model is a first machine learning model, wherein the set of image data is a first set of image data, and wherein the processing circuitry is further configured to: obtain a second set of image data generated by the image sensor before centrifugation of the set of samples, wherein the second set of image data is representative of the set of samples; and for each respective sample of the set of samples: obtain identification information associated with the respective sample based on a respective identification element associated with the respective sample; apply a second machine learning model configured to generate, based on image data representative of the respective sample in the second set of image data, a confirmation score for the respective sample, wherein the confirmation score for the respective sample is indicative of a likelihood of the identification information associated with the respective sample being accurate; determine whether the confirmation score for the respective sample satisfies a confirmation condition; and output a notification indicating whether the confirmation score for the respective sample satisfies the confirmation condition.

Example 17: The centrifuge of example 16, wherein the processing circuitry is further configured to terminate centrifugation of the set of samples in response to determining that one or more of the respective confirmation scores do not satisfy the confirmation condition.

Example 18: The centrifuge of any of examples 12 through 17, wherein the centrifuge further includes an ejection mechanism configured to eject any sample of the set of samples, and wherein the processing circuitry is further configured to control the ejection mechanism to eject a particular sample of the set of samples from the chamber in response to determining that the respective viability score for the particular sample satisfies the viability condition.

Example 19: The centrifuge of any of examples 1 through 18, wherein, for each sample of the set of samples, the viability score is based, at least in part, on a degree of region separation of the respective sample, and wherein the processing circuitry is further configured to: increase a duration of centrifugation of a particular sample of the set of samples in response to determining that the viability score for the particular sample does not satisfy the viability condition because the degree of region separation of the particular sample is low.

Example 20: The centrifuge of any of examples 12 through 19, further including a lighting element configured to emit light within the chamber at specific times, wherein the image sensor is configured to generate image data at the specific times.

For processes, apparatuses, and other examples or illustrations described herein, including in any flowcharts or flow diagrams, certain operations, acts, steps, or events included in any of the techniques described herein may be performed in a different sequence, may be added, merged, or left out altogether (e.g., not all described acts or events are necessary for the practice of the techniques). Moreover, in certain examples, operations, acts, steps, or events may be performed concurrently, e.g., through multi-threaded processing, interrupt processing, or multiple processors, rather than sequentially. Further certain operations, acts, steps, or events may be performed automatically even if not specifically identified as being performed automatically. Also, certain operations, acts, steps, or events described as being performed automatically may be alternatively not performed automatically, but rather, such operations, acts, steps, or events may be, in some examples, performed in response to input or another event.

Further, certain operations, techniques, features, and/or functions may be described herein as being performed by specific components, devices, and/or modules. In other examples, such operations, techniques, features, and/or functions may be performed by different components, devices, or modules. Accordingly, some operations, techniques, features, and/or functions that may be described herein as being attributed to one or more components, devices, or modules may, in other examples, be attributed to other components, devices, and/or modules, even if not specifically described herein in such a manner.

In one or more examples, the functions described may be implemented in hardware, software, firmware, or any combination thereof. If implemented in software, the functions may be stored on or transmitted over a computer-readable medium as one or more instructions or code and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that may be accessed by one or more computers or one or more processors to retrieve instructions, code and/or data structures for implementation of the techniques described in this disclosure. A computer program product may include a computer-readable medium.

By way of example, and not limitation, such computer-readable storage media may include RAM, ROM, EEPROM, CD-ROM, or other optical disk storage, magnetic disk storage, or other magnetic storage devices, flash memory, or any other medium that may be used to store desired program code in the form of instructions or data structures and that may be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transitory media, but are instead directed to non-transitory, tangible storage media. Disk and disc, as used herein, includes compact disc (CD), laser disc, optical disc, digital versatile disc (DVD), and Blu-ray disc, where disks usually reproduce data magnetically, while discs reproduce data optically with lasers. Combinations of the above should also be included within the scope of computer-readable media.

Instructions may be executed by one or more processors, such as one or more DSPs, general purpose microprocessors, ASICs, FPGAs, or other equivalent integrated or discrete logic circuitry, as well as any combination of such components. Accordingly, the term “processor,” as used herein, may refer to any of the foregoing structures or any other structure suitable for implementation of the techniques described herein. In addition, in some aspects, the functionality described herein may be provided within dedicated hardware and/or software modules. Also, the techniques could be fully implemented in one or more circuits or logic elements.

The techniques of this disclosure may be implemented in a wide variety of devices or apparatuses, including a wireless communication device or wireless handset, a microprocessor, an integrated circuit (IC) or a set of ICs (e.g., a chip set). Various components, modules, or units are described in this disclosure to emphasize functional aspects of devices configured to perform the disclosed techniques, but do not necessarily require realization by different hardware units. Rather, as described above, various units may be combined in a hardware unit or provided by a collection of interoperative hardware units, including one or more processors as described above, in conjunction with suitable software and/or firmware.

What is claimed is:

1. A method comprising:
initiating, by processing circuitry, centrifugation of a set of one or more samples within a chamber of a centrifuge and about a central axis;
obtaining, by the processing circuitry, a set of image data generated by an image sensor during centrifugation of the set of samples, wherein the set of image data is representative of the set of samples; and
for each respective sample of the set of samples:
applying, by processing circuitry, a machine learning model configured to generate, based on image data representative of the respective sample in the set of image data, a viability score for the respective sample, wherein the viability score is indicative of a likelihood of the respective sample being viable for an intended use;
determining, by the processing circuitry, whether the viability score for the sample satisfies a viability condition; and
outputting, by the processing circuitry, a notification indicating whether the viability score for the sample satisfies the viability condition.

2. The method of claim 1, wherein the viability score for the sample satisfies the viability condition when the viability score is equal to or greater than a threshold value.

3. The method of claim 1, further comprising controlling, by the processing circuitry, a retention assembly of the centrifuge to adjust a center of gravity of the set of samples relative to the central axis.

4. The method of claim 3, wherein controlling the retention assembly comprises rotating, by the processing circuitry, at least one of an inner ring or an outer ring of the retention assembly about the central axis, wherein the inner ring and the outer ring are each configured to retain one or more samples of the set of samples.

5. The method of claim 1, further comprising controlling, by the processing circuitry, a visual indicator of the centrifuge to indicate a respective position for each sample of the set of samples.

6. The method of claim 1, further comprising:
for each respective sample of the set of samples, obtaining, by the processing circuitry, identification information associated with the respective sample based on a respective identification element associated with the respective sample, wherein the identification information associated with the respective sample comprises at least one of a patient name, intended use, or a sample weight.

7. The method of claim 1, wherein the machine learning model is a first machine learning model, wherein the set of image data is a first set of image data, and wherein the method further comprises:
obtaining, by the processing circuitry, a second set of image data generated by the image sensor before centrifugation of the set of samples, wherein the second set of image data is representative of the set of samples; and
for each respective sample of the set of samples:
obtaining, by the processing circuitry, identification information associated with the respective sample based on a respective identification element associated with the respective sample;
applying, by processing circuitry, a second machine learning model configured to generate, based on image data representative of the respective sample in the second set of image data, a confirmation score for the respective sample, wherein the confirmation score for the respective sample is indicative of a likelihood of the identification information associated with the respective sample being accurate;
determining, by the processing circuitry, whether the confirmation score for the respective sample satisfies a confirmation condition; and
outputting, by the processing circuitry, a notification indicating whether the confirmation score for the respective sample satisfies the confirmation condition.

8. The method of claim 7, further comprising terminating, by the processing circuitry, centrifugation of the set of samples in response to the processing circuitry determining that one or more of the respective confirmation scores do not satisfy the confirmation condition.

9. The method of claim 1, further comprising controlling, by the processing circuitry an ejection mechanism of the centrifuge to eject a particular sample of the set of samples from the chamber in response to the processing circuitry determining that the respective viability score for the particular sample satisfies the viability condition.

10. The method of claim 8, wherein the ejection mechanism ejects the particular sample proximate a thermal element configured to heat or cool the particular sample to regulate a temperature of the particular sample.

11. The method of claim 1, wherein, for each respective sample of the set of samples, the viability score for the respective sample is based, at least in part, on a degree of region separation of the respective sample, and wherein the method further comprises:
increasing a duration of centrifugation of a particular sample of the set of samples in response to the processing circuitry determining that the viability score for the particular sample does not satisfy the viability condition because the degree of region separation of the particular sample is low.

12. A centrifuge comprising:
a chamber configured to contain a set of one or more samples;
an image sensor configured to generate image data; and
processing circuitry configured to:
initiate centrifugation of the set of samples about a central axis;
obtain a set of image data generated by the image sensor during centrifugation of the set of samples, wherein the set of image data is representative of the set of samples; and
for each respective sample of the set of samples:
apply a machine learning model configured to generate, based on image data representative of the respective sample in the set of image data, a viability score for the respective sample, wherein the viability score is indicative of a likelihood of the respective sample being viable for an intended use;
determine whether the viability score for the respective sample satisfies a viability condition; and
output a notification indicating whether the viability score for the respective sample satisfies the viability condition.

13. The centrifuge of claim 12, wherein the centrifuge further comprises a retention assembly, and wherein the processing circuitry is further configured to control the retention assembly to adjust a center of gravity of the set of samples relative to the central axis.

14. The centrifuge of claim 12, wherein the centrifuge further comprises a visual indicator, and wherein the processing circuitry is further configured to control the visual indicator to indicate a respective position for each sample of the set of samples.

15. The centrifuge of claim 12, wherein the processing circuitry is further configured to, for each respective sample of the set of samples, obtain identification information associated with the respective sample based on a respective identification element associated with the respective sample, wherein the identification information associated with the respective sample comprises at least one of a patient name, intended use, or a sample weight.

16. The centrifuge of claim 12, wherein the machine learning model is a first machine learning model, wherein the set of image data is a first set of image data, and wherein the processing circuitry is further configured to:
obtain a second set of image data generated by the image sensor before centrifugation of the set of samples, wherein the second set of image data is representative of the set of samples; and
for each respective sample of the set of samples:
obtain identification information associated with the respective sample based on a respective identification element associated with the respective sample;
apply a second machine learning model configured to generate, based on image data representative of the respective sample in the second set of image data, a confirmation score for the respective sample, wherein the confirmation score for the respective sample is indicative of a likelihood of the identification information associated with the respective sample being accurate;
determine whether the confirmation score for the respective sample satisfies a confirmation condition; and
output a notification indicating whether the confirmation score for the respective sample satisfies the confirmation condition.

17. The centrifuge of claim 16, wherein the processing circuitry is further configured to terminate centrifugation of the set of samples in response to determining that one or more of the respective confirmation scores do not satisfy the confirmation condition.

18. The centrifuge of claim 12, wherein the centrifuge further comprises an ejection mechanism configured to eject any sample of the set of samples, and wherein the processing circuitry is further configured to control the ejection mechanism to eject a particular sample of the set of samples from the chamber in response to determining that the respective viability score for the particular sample satisfies the viability condition.

19. The centrifuge of claim 1, wherein, for each sample of the set of samples, the viability score is based, at least in part, on a degree of region separation of the respective sample, and wherein the processing circuitry is further configured to:
increase a duration of centrifugation of a particular sample of the set of samples in response to determining that the viability score for the particular sample does not satisfy the viability condition because the degree of region separation of the particular sample is low.

20. The centrifuge of claim 12, further comprising a lighting element configured to emit light within the chamber at specific times, wherein the image sensor is configured to generate image data at the specific times.
